(19) United States
(12) Patent Application Publication
(10) Pub. No.: US 2013/0102069 A1
(43) Pub. Date: Apr. 25, 2013

(54) ALGAE REACTOR

(71) Applicant: Tendris Solutions B.V., Almere (NL)

(72) Inventors: Taco Wijnand NEEB, ALMERE (NL); Johannes Otto ROOYMANS, ERMELO (NL)

(73) Assignee: TENDRIS SOLUTIONS B.V., ALMERE (NL)

(21) Appl. No.: 13/707,685

(22) Filed: Dec. 7, 2012

Related U.S. Application Data
(62) Division of application No. 13/360,834, filed on Jan. 30, 2012.
Application No. 13/360,834 is a continuation of application No. PCT/EP2010/061153, filed on Jul. 30, 2010.
(60) Provisional application No. 61/229,806, filed on Jul. 30, 2009.

(51) Int. Cl. C12M 1/00; F21V 7/00; F21V 21/00

(52) U.S. Cl. CPC C12M 21/02; F21V 21/00; F21V 7/00
USPC 435/292.1; 362/249.02; 362/235

(57) ABSTRACT

The invention relates to a method for transferring light generated by a light emitting diode towards an aqueous liquid containing algae. The method includes emitting light by the light emitting diode having a first refractive index, transferring the light through a liquid medium having a second refractive index, and a solid medium having a third refractive index, into the aqueous liquid having a fourth refractive index. The value of the first refractive index is equal to or greater than the value of the second refractive index. The value of the second refractive index is equal to or greater than the value of the third refractive index. The value of the third refractive index is equal to or greater than the value of the fourth refractive index. The invention further relates to a reactor designed to perform such method.

### CROSS-REFERENCE TO RELATED APPLICATIONS

This application is a division of U.S. application Ser. No. 13/360,834 filed on Jan. 30, 2012, which is a continuation of PCT application number PCT/EP2010/061153, filed on Jul. 30, 2010, which claims priority from U.S. provisional application No. 61/229,806, filed on Jul. 30, 2009. The contents of all of these applications are hereby incorporated by reference in their entirety.

### BACKGROUND OF THE INVENTION

1. Field of the Invention

The invention relates to a bioreactor for growing algae in an aqueous liquid using photosynthesis. In particular, the invention relates to a lighting system for such bioreactor. The present invention further relates to a method for growing algae, and a method of providing lighting for the algae.

2. Description of the Related Art

The photosynthesis process is conversion of light energy into chemical energy by living organisms, such as algae. The raw materials are carbon dioxide and water; the energy source is light; and the end-products are oxygen and (energy rich) carbohydrates. Algae have been recognized as an efficient producer of biomass, and in particular oil from which biodiesel and other fuels can be produced. During photosynthesis, algae absorb carbon dioxide (CO₂) and light (photons) in the presence of water and produce oxygen and biomass. Dissolved nutrients may assist the process. Algae can produce lipids or vegetable oils which can be converted into biodiesel and other biofuels or used directly.

The benefits of using algae to efficiently grow biomass and produce biofuel have been known for a long time, and various methods have been used to grow algae in laboratories and small scale experimental units. However, it has proven difficult to grow algae efficiently on a commercial scale.

Open pond systems have been used to grow algae on a large scale. These systems are not very efficient. In open pond systems it is difficult to control temperature and pH, and difficult to prevent foreign algae and bacteria from invading the pond and competing with the desired algae culture. Furthermore, much of the sunlight is reflected by the water's surface, and the sunlight that does enter the pond only penetrates a small distance into the water due to the algae becoming so dense and blocking the light, so that the sunlight only reaches a thin layer of algae growing near the surface of the pond.

Bioreactors have also been used, in which nutrient-laden water is pumped through plastic or glass tubes or plates that are exposed to sunlight. Such bioreactors are more costly and more difficult to operate than open pond systems, and they also suffer from the problem of getting the sunlight to the algae where it can be absorbed. A large portion of the sunlight is reflected from the surface of the tubes or plates. Only a small amount of the sunlight enters the water in the tubes or plates, and this small amount of sunlight only penetrates a small distance into the volume of the tube or plate. Other drawbacks of such bioreactor systems are the difficulty of temperature control, and the reliance on sunlight for growing the culture.

Algae grows best under controlled conditions. Algae is sensitive to temperature and light conditions. By controlling all aspects of the cultivation, such as temperature, CO₂ levels, light and nutrients, extremely high yields can be obtained.

### BRIEF SUMMARY OF THE INVENTION

The present invention aims to provide an improved bioreactor using a light emitting diode (LED) lighting system to at least partially provide the light for the algae.

For this purpose, embodiments of the invention relate to a lighting system for illuminating algae in an aqueous liquid comprising a light source comprising a plurality of LEDs, a mounting structure for supporting the LEDs, and a housing for accommodating the light source and the mounting structure, at least a portion of the housing being transparent for light emitted by the light source, wherein the housing is at least partly filled with a cooling liquid, such that, in use, heat from the LEDs is transferred by the cooling liquid from the LEDs by means of convection.

In one aspect, the invention relates to a reactor for growing a algae in an aqueous liquid using photosynthesis, the reactor comprising a tank for accommodating the aqueous liquid with the algae in it; and the abovementioned lighting system for illumination of the algae, wherein the lighting system is at least partially submerged in the aqueous liquid.

In another aspect, the invention relates to a method for growing algae in an aqueous liquid using photosynthesis, the method comprising: providing an aqueous liquid with the algae in it, providing a lighting system at least partially submerged in the aqueous liquid, the lighting system comprising a plurality of LEDs, providing a cooling liquid for cooling the LEDs of the lighting system, and irradiating the algae with light generated by the LEDs, the light being transmitted through the cooling liquid and into the aqueous liquid in a region below the top surface of the aqueous liquid.

In yet another aspect, the invention relates to a method for transferring light generated by a light emitting diode towards an aqueous liquid comprising algae, the method comprising: emitting light by the light emitting diode, the light emitting diode having a first refractive index; transferring the light through a medium having a second refractive index; further transferring the light through a solid medium having a third refractive index; and passing the light into the aqueous liquid, the aqueous liquid having a fourth refractive index; wherein the values of the first, second, third and fourth refractive index form a sequence with a descending order.

### BRIEF DESCRIPTION OF THE DRAWINGS

Various aspects of the invention will be further explained with reference to embodiments shown in the drawings wherein:

FIG. 1A is a simplified top view of an embodiment of a bioreactor with lighting systems;

FIG. 1B is a perspective view of the bioreactor of FIG. 1A;

FIG. 2 is a perspective view of an embodiment of a lighting system;

FIG. 3A is a simplified top view of an arrangement of LEDs in a lighting system;

FIG. 3B is a simplified top view of another arrangement of LEDs in a lighting system;

FIG. 4A is a cross-sectional view of a two-sided mounting arrangement for LEDs;

FIG. 4B is a cross-sectional view of a one-sided mounting arrangement for LEDs;

FIG. 5 is a perspective view of a mounting arrangement for LEDs;

FIG. 6 is a cross-sectional side view of a lighting system showing circulation of cooling fluids;

FIG. 7 is a cross-sectional view of a diffuser arrangement;

FIG. 8A is top view of a reflector arrangement for LEDs;

FIG. 8B is a cross-sectional view of a reflector arrangement for LEDs;

FIGS. 8C, 8D are different perspective views of a reflector arrangement for LEDs;

FIG. 9 is a cross-sectional view of an alternative arrangement of a lighting system;

FIG. 10 is a cross-sectional view of another alternative arrangement of a lighting system with a transparent top portion;

FIG. 11 is a side view and top cross-sectional view of an alternative lighting system having a tubular housing;

FIG. 12 is a perspective view of the lighting system of FIG. 11 partially dismantled;

FIG. 13 is a cross-sectional view of the lighting system of FIG. 11;

FIG. 14 is simplified schematic diagram of a bioreactor with lighting systems having tubular housings;

FIG. 15A is a cross-sectional view of a disc pump for a bioreactor; and

FIG. 15B is another cross-sectional view of the disc pump of FIG. 15A.

### DESCRIPTION OF ILLUSTRATIVE EMBODIMENTS

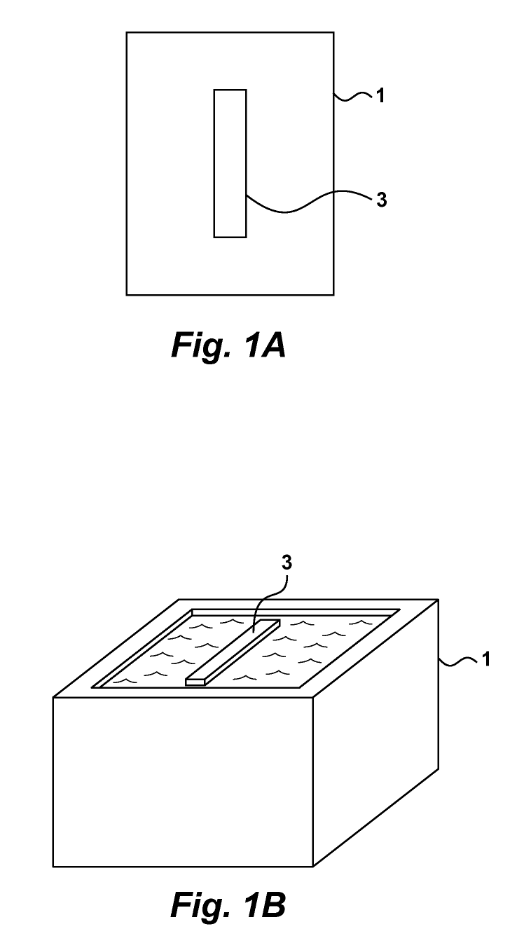

The following is a description of various embodiments of the invention, given by way of example only and with reference to the drawings. FIG. 1A is a simplified top view of an embodiment of a bioreactor with lighting systems, and FIG. 1B is a perspective view of the bioreactor. The bioreactor comprises a tank 1 containing an aqueous liquid in which algae is grown. The aqueous liquid may be fresh water or salt water or some other suitable aqueous solution, but for simplicity is referred to herein as water. The expression “algae” should be understood to include algae, cyanobacteria or any other suitable photosynthetic organism capable of growing using photosynthesis. For simplicity throughout the specification the expression “algae” is used.

The lighting systems 3 are at least partially submerged in the water. This enables much more of the light emitted from the lighting system to be transmitted into the water, by emitting the light from the walls of the lighting system at a point below the top surface of the water. The use of lighting systems submerged in the water permits improved and more flexible transmission of light into the water by arranging the lighting systems closely enough so that the light reaches most of all of the algae in the volume of water in the tank.

The use of artificial light inside the bioreactor tank avoids the need to construct the tank from a transparent material. This reduces cost and enables the bioreactor tank to be made from cheaper and more durable materials, and results in tanks that are more easily fabricated. The bioreactor tank may be made, for example, from steel, stainless steel, and the like.

The tanks may also be much taller than a pond or traditional bioreactor dependent on sunlight. This enables tanks to have a much smaller footprint for the same volume of algae culture, saving ground space and enabling a much more compact algae growth facility. This has particular importance in urban environments or where land costs are high.

Accurate temperature control of the water in the tank is also more easily achieved with the bioreactor of FIGS. 1A, 1B. A bioreactor relying on exposure to sunlight requires a large surface area. A more compact arrangement with less surface area reduces the effect of outside temperature variations, and non-transparent tank walls reduce the temperature variation due to variations temperature and sunlight from day to night and summer to winter.

Accurate control of the light received by the algae is also more easily achieved with the bioreactor of FIGS. 1A, 1B. Ponds or bioreactors relying on sunlight are subject to wide variations in light exposure, between night and day, sunny or cloudy conditions, long summer days or short winter days. By using artificial light, the light exposure period is increased to 24 hours per day, and constant lighting is provided throughout the year regardless of outside conditions. The lighting system can be tailored to provide light in the specific wavelengths which can be used by the algae for growth. The lighting system can also be tailored to provide light at the right intensity to achieve high growth rates, while avoiding excessive exposure which harms the algae.

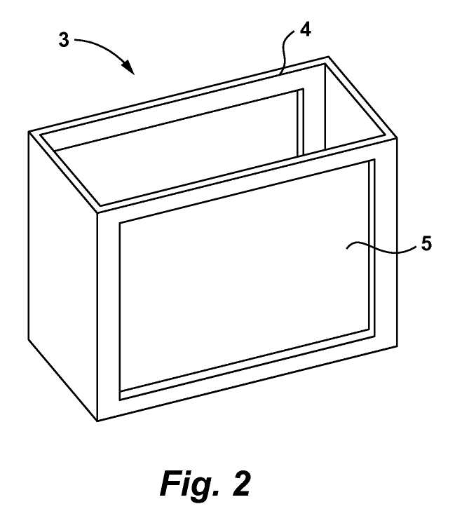

FIG. 2 is a perspective view of an embodiment of the lighting system 3. The lighting system 3 has a housing comprising a frame 4 with transparent walls 5. Alternatively, the frame itself can be constructed of a suitable transparent material. The transparent walls 5 may be made of glass, polycarbonate, or other suitably strong transparent material. Preferably, the transparent material like glass has a refractive index of 1.3 or higher.

The lighting system 3 may comprise an arrangement of LEDs 20. The expression LEDs in this context also refers to LED chips or LED dies. The LEDs 20 may be mounted on a ceramic carrier like a ceramic printed circuit board (PCB), which is mounted on a mounting structure within the lighting system 3. Preferably, the mounting structure is a planar structure. The ceramic carrier may be a metal core PCB to support a large number of LEDs, for example 60 LEDs. The ceramic carrier with naked bonded LEDs may be glued or eutectic bonded on the mounting structure.

The LEDs 20 form a light source for illuminating or irradiating the algae in the bioreactor tank 1. The light intensity of the light source can be tailored to be of sufficient intensity to substantially prevent growth of the algae on the surface of the transparent portion of the housing. The light source may comprise different types of LEDs, emitting light in certain specific wavelengths most suited to promoting growth of the algae. For example, the light source may comprise a combination of one or more LEDs for emitting light with a wavelength in the range of 400-500 nm, preferably 400-450 nm (e.g. blue LEDs) and one or more LED for emitting light with a wavelength in the range of 600-685 nm, preferably 640-670 nm (e.g. red LEDs). The LEDs for emitting 640-670 nm light may be an aluminum indium gallium phosphide LED.

In some embodiments, the light source is arranged so that, in operation, most of the light emitted from the light source has a wavelength in the ranges of 400-450 nm and 640-670 nm, preferably 80% or more. These wavelengths are chosen to match the absorption maxima of chlorophyll and the pigments which are used by various types of algae to grow.

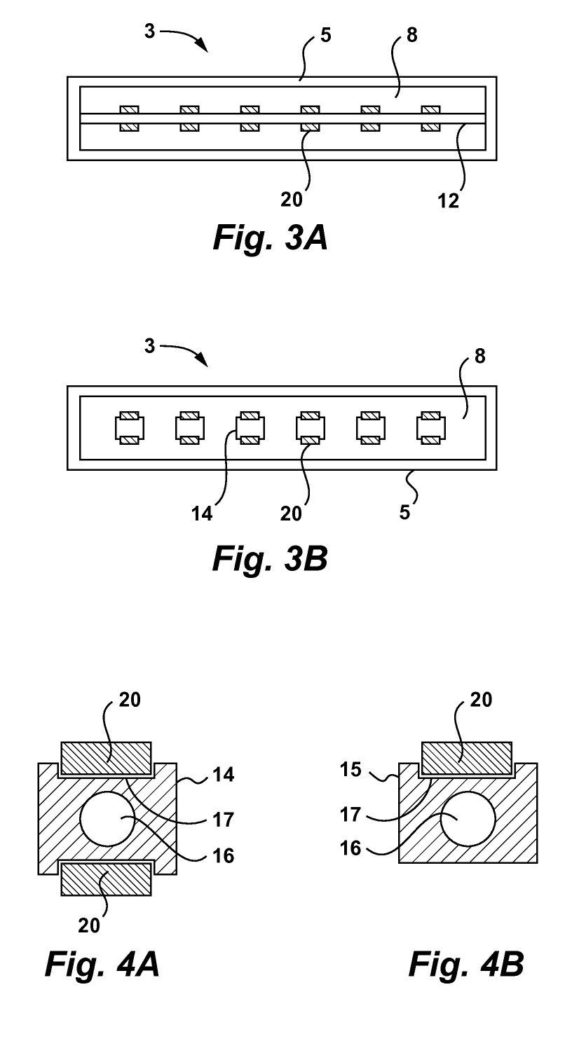

FIG. 3A is a simplified top view of an arrangement of LEDs in the lighting system 3. A mounting plate 12 is arranged in a vertical position in the interior space 8 of the lighting system 3. LEDs 20 are arranged on the plate to emit light through the transparent walls 5. The mounting plate 12 is preferably rigid and a good heat conductor, such as aluminum, copper or steel, to conduct heat away from the LEDs which get hot during operation. The interior space 8 may be filled with a cooling liquid 19 in direct contact with the LEDs to transfer heat away from the LEDs. Additionally or alternatively, the plate 12 may be provided with one or more cooling channels for circulation of a second cooling fluid for enhancing the removal of heat from the LEDs. FIG. 3B shows an alternative arrangement of LEDs mounted on mounting struts 14 arranged vertically in the lighting system.

FIG. 4A shows a cross-section of a two-sided mounting arrangement. The mounting strut 14 has an internal channel 16 for circulation of a cooling fluid for cooling the LEDs. The mounting strut may also include a recess 17 on each side in which the LEDs 20 are mounted. This design permits secure mounting of the LEDs which face outwards to emit the maximum light towards the transparent walls 5 on each side of the lighting system, while providing cooling to the back of the LEDs. The mounting struts are preferably made from a good heat conductor, such as aluminum or copper, to efficiently conduct heat away from the LEDs. FIG. 4B shows an alternative one-sided mounting arrangement for LEDs.

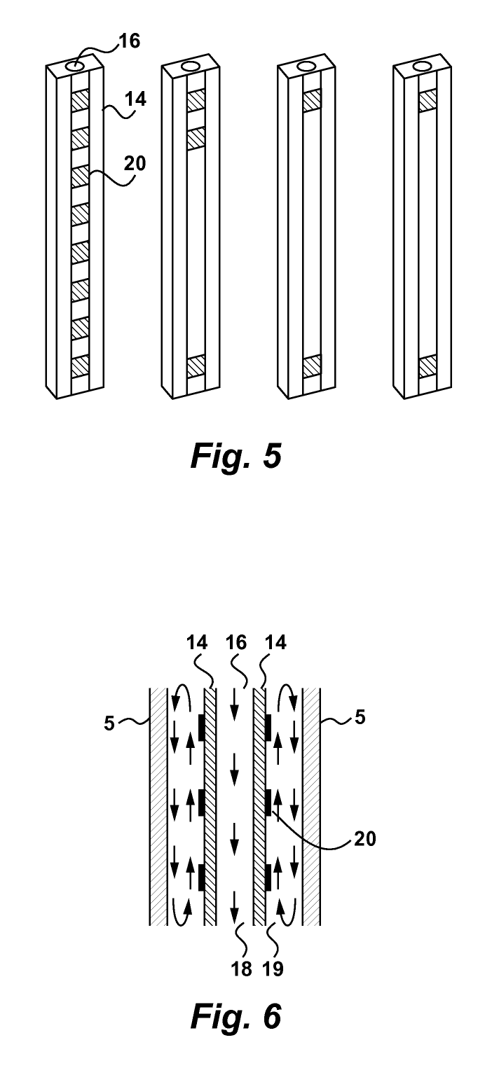

FIG. 5 shows a perspective view of mounting struts 14 arranged vertically side-by-side along the length of the lighting system. The use of mounting struts 14 in a vertical arrangement allows for a more flexible modular construction of the lighting system, which may be beneficial in terms of flexibility and capability to match the lighting system requirements with the algae species to be illuminated.

FIG. 6 shows a cross-section through the lighting system and mounting strut showing circulation of the two cooling fluids for cooling the LEDs. The LEDs 20 are mounted on both sides of mounting strut 14, with channel 16 formed in the mounting strut.

All of the above embodiments may use two cooling fluids, a first cooling liquid in direct contact with the front side of the LEDs and a second cooling fluid flowing in a channel to remove heat from the back side of the LEDs.

The first cooling liquid 19 fills the interior space 8 between the LEDs 20 and the transparent wall 5 of the lighting system. This cooling fluid flows past the external front surface of the LEDs, preferably in direct contact with the LEDs. The cooling liquid 19 is preferably an oil. The cooling liquid 19 preferably circulates under natural convection, rising from the bottom of the lighting system as it gets hotter from contact with the LEDs. The LED chips are preferably mounted vertically, with the LED's bottom electrode against the mounting plate 12 or mounting strut 14 to promote heat transfer from the LED to the mounting structure. The LED's top electrode faces outwards and is cooled by the cooling liquid 19. The LED dies may be provided with a very thin protection or passivation film, to provide physical protection while still permitting good heat transfer from the LEDs to the cooling liquid. The blue LEDs (emitting in the range 400-500 nm, preferably 400-450 nm) preferably have a protection or passivation film, preferably only on the top surface, to protect them from the cooling liquid 19. The red LEDs (emitting in the range 600-685 nm, preferably 640-670 nm) preferably do not have any protection or passivation film, as they are not affected by the cooling liquid.

Forced convection of the cooling liquid 19 may also be used, although excessive flow may damage the bond wires of the vertically arranged LEDs. Furthermore, for this reason, the bond wires of the LEDs 20 preferably extend in a direction parallel to the flow of cooling liquid 19.

The first cooling liquid 19 is preferably an oil with a high refractive index, such as Dow Corning C5 or C51. The lighting system is preferably constructed of materials selected to have favorable refractive indices to maximize the transmission of light from the LEDs into the water containing the algae. The LED chips typically have a refractive index of about 3.3 for red LEDs and 2.2. for blue LEDs. It is advantageous if the first cooling liquid is in direct contact with the LED and has a refractive index matching the LED as closely as possible. This reduces reflection of light at the boundary between the LED 20 and the cooling liquid 19 to result in the maximum extraction of photons from the LEDs.

A suitable cooling liquid 19 has a refractive index, good transparency, and sufficiently low viscosity to flow easily over the LEDs under natural convection. The first cooling liquid 19 preferably has a refractive index in the range of 1.5 to 1.7, and preferably up to 1.62. Highly refractive titanium dioxide (TiO₂) nano particles, preferably with a refractive index of about 1.8, may be dissolved in the cooling liquid 19 to increase the refractive index of the suspension to about 1.7.

The first cooling liquid 19 also has other advantages. The film of cooling liquid/oil 19 ensures good thermal contact between the LEDs 20, mounting structure 12 or 14, and the transparent wall 5. Wetting of the LED chip's front surface by the cooling liquid 19 improves heat transfer from the LEDs. A suitable cooling liquid 19 also acts to reduce deterioration of the encapsulant of the LEDs. The cooling liquid 19 also enables thinner transparent walls to be used for the lighting system, especially for deep lighting systems placed in deep water (e.g. 2 m or more) in tall bioreactor tanks, since the cooling liquid pressurizes the interior to the lighting system to assist in counteracting the external pressure from the water.

The second cooling fluid 18 may be circulated in channels behind the LEDs in the mounting plate 12 or mounting struts 14 to increase the cooling capacity of the system. The cooling fluid 18 may be water, preferably water that has not been in contact with the water in the bioreactor tank 1. In a preferred embodiment, the cooling fluid has a temperature below 0° C. In such case the cooling liquid 18 may be a refrigerant or a cooled gas, for example cooled carbon dioxide gas. Cooling the LEDs via the channel 16 with a cooling fluid at a relatively low temperature, e.g. below 10° C., preferably below 0° C., enables the LEDs to operate at a relatively low temperature as well, which will increase the performance of the LEDs 20. Additionally, the possibility to choose the type of cooling fluid 18 may help to adjust the temperature of the water in the bioreactor to a temperature that suits a specific species of algae.

In FIG. 6, the second cooling fluid 18 is circulated in the channel 16 is directed in a direction opposite to the direction of the convective flow of the first cooling liquid 19. Although this arrangement is preferred, it is also possible that the second cooling fluid 18 travels through the channel 16 in a direction that is similar to the direction of the first cooling liquid 19.

The entire construction of the submerged lighting system is preferably designed to maximize light transmission from the LEDs into the water containing the algae. This is accomplished by matching the refractive indices as closely as possible of the materials through which the light passes from the LEDs to the water containing the algae and avoiding large differences in the refractive indices of these materials. As discussed above, a first cooling liquid 19 preferably has a high refractive index to reduce reflection at the boundary between the LEDs and the cooling liquid. The transparent wall 5 is preferably constructed of a material with a refractive index that approximates or matches the first cooling liquid 19, for example glass with high lead content or any other transparent material like, for example, polycarbonate or epoxies. A typical refractive index of glass is 1.52 which can be increased by the addition of lead to match the preferred range for cooling liquid 19 of 1.5 to 1.7. Water has a refractive index of about 1.33. Thus, matching the refractive indices of the cooling liquid 19 and transparent wall 5 will reduce reflections at that boundary, but may increase reflection at the boundary between the transparent wall and the water containing the algae.

Preferably, light emitted by the LEDs does not pass through air before being emitted from the transparent portion of the housing. In such embodiment, the light solely passes through liquid and solid media before such emission. In other words, the submerged lighting system preferably has no low refractive index layer, such as air, between the LEDs and the water containing the algae. Thus, although there is a decrease of the refractive indices of the layers of material through which the light passes, there is no increase. For example, the approximate refractive indices in one embodiment may be: LED 3.3 (red LED) or 2.2 (blue LED), cooling liquid 1.7, transparent wall 1.7 (glass with lead content) or 1.52 (glass without lead) or 1.42 (polycarbonate), and water 1.33. With this arrangement, the lighting system can achieve improved coupling of light from the LEDs to the water, of 2.5 or more micromoles of photons per watt of power input to the lighting systems. In contrast, lighting systems with an air gap can only achieve values around 1.0 micromoles per watt. A bioreactor with this type of lighting arrangement can achieve algae growth resulting in a doubling of the algae every 6 hours, as opposed to previous systems relying on sunlight which typically achieve a doubling of the algae every 24 hours.

Growth of algae on the outside surface of the transparent portions of the lighting panel housing reduces the effectiveness of the lighting system. This algae adhering to the transparent walls will not circulate in the water and blocks light from the LEDs from reaching the bulk of the algae circulating in the water. This undesirable algae growth can be reduced or eliminated by adjusting the intensity of the light source. In operation, the light transmitted through the transparent walls 5 is preferably of sufficient intensity to substantially prevent growth of algae on the surface of the transparent walls. A light flux of 1000 micromoles per second per square meter or higher at the outside surface of the transparent wall has been shown to be sufficient for this purpose. The light should not be too intense to prevent harm to the algae circulating in the water.

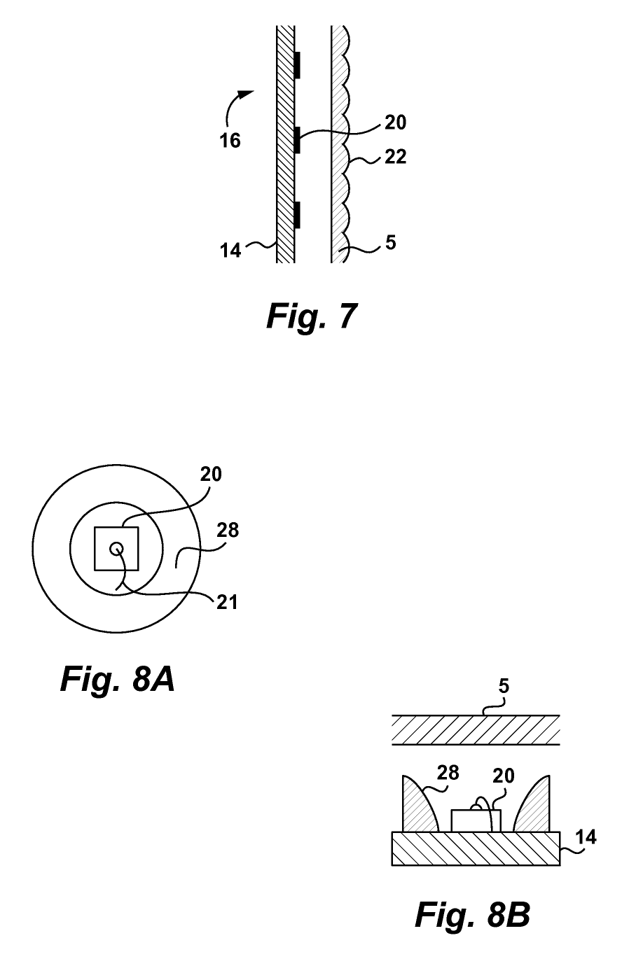

FIG. 7 is a cross-sectional view of a lighting system provided with a diffuser arrangement 22. The transparent walls 5 preferably include a diffuser arrangement 22 to disperse light from the LEDs 20 into the water. The diffuser arrangement 22 may take the form of convex shapes on the outside of the transparent walls 5 of the housing. Alternatively, or additionally, the diffuser arrangement may take the form of a diffusion film or sheet that is provided on a surface of the transparent walls of the housing.

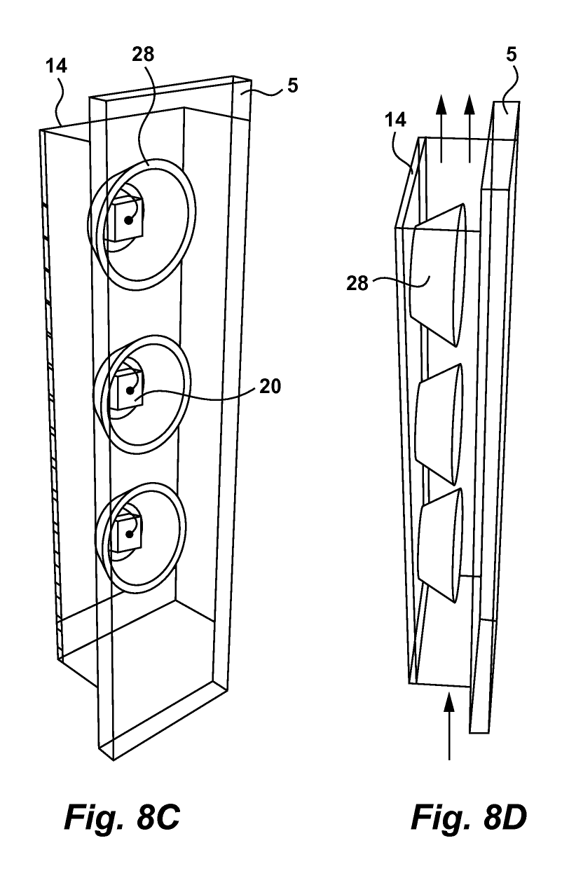

FIG. 8A is top view of a reflector arrangement that may be used in combination with one or more of the LEDs 20, while FIG. 8B shows a cross-section of a specific embodiment of such reflector arrangement. FIGS. 8C, 8D are different perspective views of a reflector arrangement that differs from the reflector arrangement of FIG. 8B. The reflector arrangement of FIGS. 8C, 8D comprises one or more reflectors 28 that may be used around the LEDs 20 to increase light transmission from the LEDs into the water, by directing light emitted from LED in direction substantially perpendicular to the transparent wall 5. The one or more reflectors 28 may take the form of concave structures surrounding the LED, for example as a rim structure as shown in FIG. 8B, 8C or 8D. The concave structures may be made of a metal, or a material with a low refractive index sufficiently different from the cooling liquid 19 to result in good reflection of light from the LEDs, preferably an easily formed material like a suitable epoxy, preferably with a refractive index of about 1.1. The rim structure can be shaped as shown in FIG. 8B to ensure that the combination of shape and refractive index of the rim structure material reflects light emitted by the LED 20. The reflector arrangement preferably comprises a circular reflecting surface surrounding each LED to enhance the uniformity of light emission.

The reflector arrangement can be designed such that it limits the angle at which light is emitted by a LED towards the water. The outer angle at which light emitted by the LEDs is received at the interface between the cooling fluid and the transparent wall may be arranged such that total reflection at this interface, and preferably also at the interface between the transparent wall and the water, are avoided as much as possible. By limiting the exit angle of the LEDs in such a way, the reflector arrangement reduces efficiency losses due to total reflection. For similar reasons, preferably, the reflector is arranged to reflect light emitted from the LEDs towards the transparent wall of the lighting system substantially at right angles to the surface of the transparent wall.

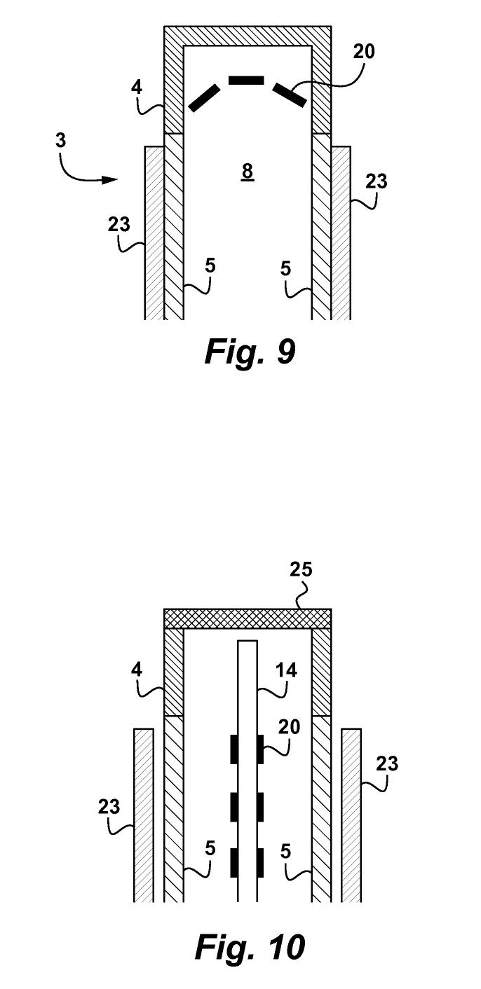

FIG. 9 is an alternative arrangement of a lighting system 3. In this arrangement, instead of mounting the LEDs 20 along a plate or strut within the lighting system 3, the LEDs 20 are mounted in the top of the frame 4 and directed inward to the lighting system 3. In this embodiment, a surface of the transparent walls 5 of the lighting system 3, preferably the outside surface, is covered with a diffusion arrangement, for example a diffusion film or sheet 23. The diffusion arrangement is arranged to diffuse the light emitted by the LEDs 20 so as to distribute the light throughout the bioreactor tank as evenly as possible.

FIG. 10 is yet another alternative arrangement of a lighting system. In this arrangement, the LEDs 20 are mounted on a mounting strut 14. The frame 4 comprises a cover structure 25 or top portion that is substantially transparent for external light, preferably sunlight. The transparent top portion 25 may comprise a reflector for (re-)directing sunlight into the housing. In an embodiment, the transparent top portion 25 comprises a filter. Such filter may filter out light with wavelengths that are considered not useful for irradiating the algae, for example because it will not be absorbed or will limit the growth of an algae. The filter may be replaceable, and may be adapted in view of the type of algae being grown.

The external light that is coupled into the lighting system via the top portion or cover structure 25 is provided to the aqueous liquid in the tank via the transparent walls 5 of the lighting system. Preferably, for similar reasons as discussed with reference to the embodiment shown in FIG. 9, the (outside) surface of the transparent walls 5 of the lighting system are provided with a diffusion film or sheet 23.

The embodiment of the lighting system of FIG. 10 has the advantage that besides light provided by LEDs, the light can be balanced by external light such as sunlight to provide the algae in the bioreactor tank with optimal light conditions. Consequently, it may be possible to obtain the same results with respect to algae growth with less energy consumption by the LEDs 20 as the external light provides an additional light flux. The external light may be collected via light collectors and reflectors and distributed throughout the lighting system in a controllable way, e.g. by using one or more of lenses, light conductors like fiber optics, and diffusion optics. Some or all of these optical elements may be included in the cover structure 25. In this way, optimal light conditions may be created per algae species.

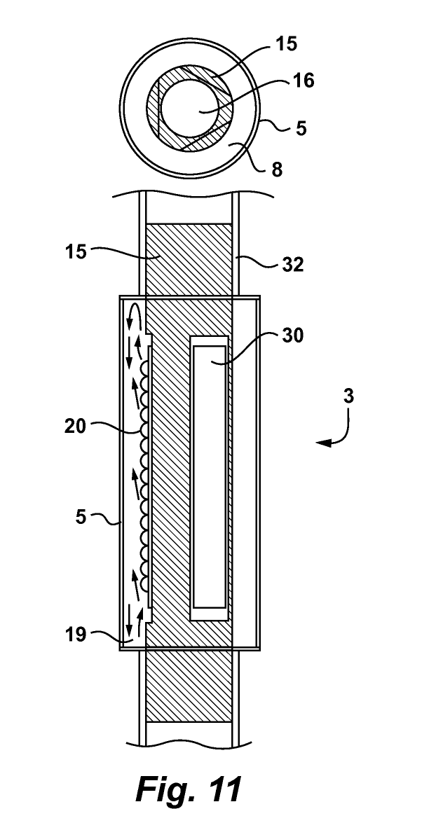

FIG. 11 is a side view and top cross-sectional view of an alternative lighting system having a tubular housing. The lighting system includes a tubular mounting structure 15 for supporting light source 30, the tubular mounting structure having an internal channel 16 for a circulation of a cooling liquid for cooling the light source.

The housing includes a transparent wall 5 in a tubular shape, the tubular mounting structure 15 and tubular transparent wall 5 being arranged concentrically. The light source 30 is formed on a planar section formed in the outer surface of the tubular mounting structure 15. The light source includes a strip of LEDs 20 mounted on a ceramic printed circuit board, which is mounted on the planar section. The ceramic carrier may be a metal core PCB to support a large number of LED chips, for example 60 chips. The ceramic carrier with naked bonded LED dies may be glued or eutectic bonded on the flat planar section of the mounting structure 15.

More than one light source 30 may be located at a certain position along the length of the tubular mounting structure. In the embodiment shown in FIG. 11, three light sources 30 are arranged at equal spacing around the circumference of the tubular mounting structure 15. The tubular mounting structure 15 may be formed in long lengths having light sources arranged at several positions along its length. The tubular mounting structure 15 may also be constructed in shorter lengths and joined to other mounting structures using a connecting sleeve 32.

An interior cavity 8 is formed in the gap between the two tubes of the mounting structure 15 and the transparent wall 5, the cavity filled with a cooling liquid 19, preferably oil with a high refractive index. In one embodiment the amount of oil for this small cavity is minimal. The small quantity of cooling liquid results in minimal circulation of the oil in the cavity 8, which reduces the chance of damage to the bond wire or LED chips and reduces damage or wear and tear caused by any particles of pollution in the cooling liquid.

In another embodiment there is sufficient cooling liquid in the cavity 8 to result in natural convection current in the cooling liquid to enhance the transfer of heat away from the LEDs. The lighting system is preferably disposed with its longitudinal axis in a vertical direction to provide a sufficient vertical distance over the length of the light sources 30 to promote the natural convection current within the cooling liquid 19.

The same materials may be used for this embodiment of the lighting system as the previous embodiment of FIG. 2, for the transparent wall, mounting structure, cooling liquids etc. The materials used for this embodiment preferably have refractive indices that result in maximizing the light coupling between the LEDs and the water containing the algae, as discussed for the previous embodiments. The same considerations apply for this embodiment and for the previous embodiments. A high refractive index cooling liquid has a positive effect on the light out-coupling from the LEDs to the water/algae, and wetting of the LED chip surface to improve heat transfer. The cooling liquid may also reduce problems of deteriorating encapsulant of the LEDs. A thin film of cooling liquid will also get around the whole tube, ensuring optimal thermal contact between the mounting structure 15 and the transparent wall 5. The cooling liquid may also prevent any electrolyze effects on the light source and connections. The connection wires and electronics to provide a constant driving current to the LEDs can be integrated on the same mounting structure 15 on a flat section of the tube.

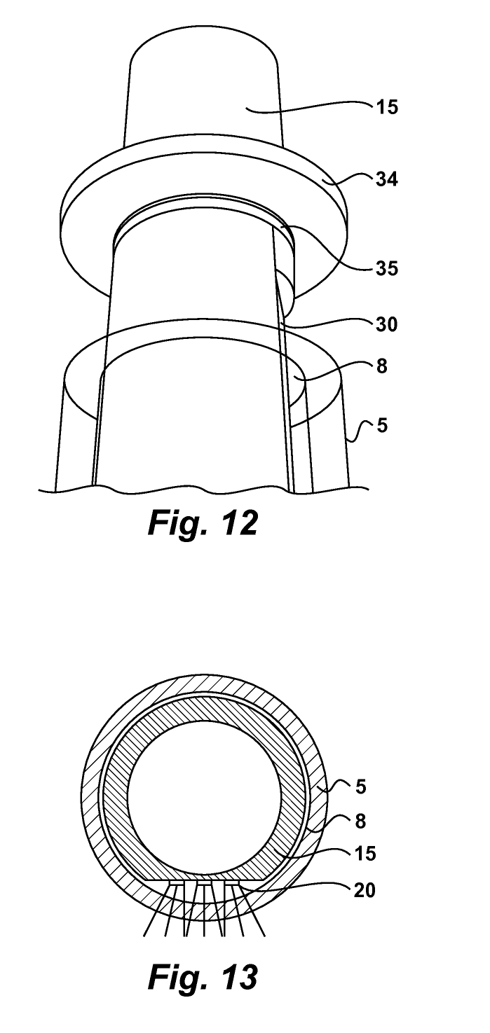

FIG. 12 is a perspective view of the lighting system of FIG. 11 partially dismantled to show the end cap 34 and sealing ring 35 for sealing off the ends of the cavity 8 formed between the mounting structure 15 and transparent wall 5. The end cap 34 and sealing ring 35 function to separate the cavity 8 from the water of the bioreactor, to keep the cooling liquid from leaking from the cavity and prevent water from entering the cavity. The initial filling of the cavity 8 with the cooling liquid 19 can be done with a thick injection needle through the hole between the transparent wall and the flat planar section of the mounting structure. The transparent wall can then be moved up over the rubber sealing ring 35 and the last part of the oil can be filled through this ring with a thin injection needle.

FIG. 13 is a cross-sectional view of the lighting system showing the flat planar portion of the tubular mounting structure 15 where the LEDs 20 of the light sources are located.

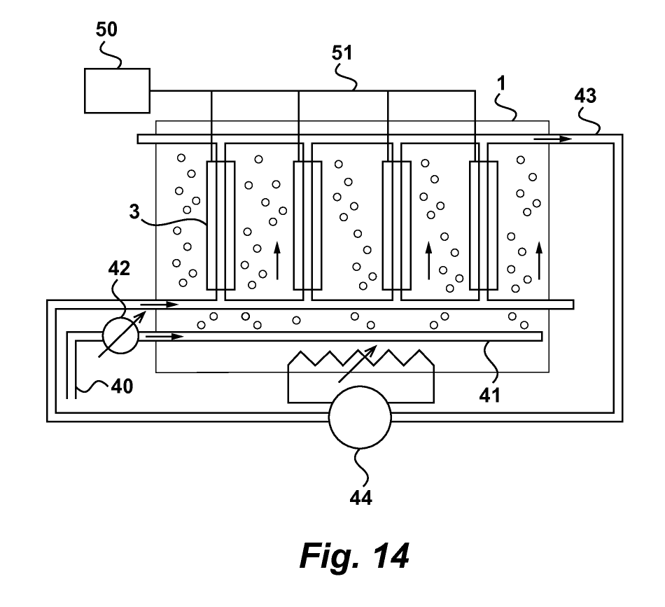

FIG. 14 is simplified schematic diagram of a bioreactor with lighting systems 3 comprising a number of LED lamps. The LED lamps may be accommodated in the light systems as described with reference to FIGS. 3A, 3B or they may be accommodated by tubular housings as described with reference to FIGS. 11-13.

The bioreactor may comprise a CO₂ supply system 40 including a CO₂ supply device 41 to supply carbon dioxide (CO₂) to the water containing the algae. Preferably, in an embodiment of a bioreactor tank 1 which comprises a CO₂ supply, the LEDs 20 are arranged vertically, for example as shown in FIG. 5 or 11, to provide a consistent light level as CO₂ rises through the water.

A cooling fluid is supplied to the LED light source via a separate cooling fluid supply system 43. The cooling fluid corresponds to the second cooling fluid 18 discussed above. The bioreactor further comprises a heater 42 for heating the CO₂ before it is supplied to the bioreactor tank in the form of CO₂ gas, schematically represented by bubbles in FIG. 14. The bioreactor further comprises a heat exchanger 44 for cooling the cooling fluid. The heat exchanger is arranged to remove heat from the cooling fluid after passage through the lighting systems 3 in the bioreactor, and to supply the heat removed from the cooling fluid to the water containing the algae, and/or a heater for heating the CO₂ supplied to the bioreactor, and/or another medium to remove the heat from the system. The reuse of heat from the cooling liquid 18 allows for a bioreactor with a very efficient performance.

It is preferable that the temperature of the LEDs and the temperature of the water containing the algae are under separate control. Although the heat exchanger may reuse heat from the cooling fluid 18 to heat the water or injected CO₂, it is preferable that separate control of the cooling fluid temperature and the water temperature is maintained.

The bioreactor also comprises a control system 50 for supplying power to the LED lighting system. Carbon fixation in algae, which is part of the photosynthesis process, occurs in the dark. The control system may cycle the LEDs rapidly on and off to increase carbon fixation in the algae and increase the growth rate of the algae, for example switching the LEDs on and off in a cycle of 10 milliseconds on and 10 milliseconds off. The electrical connections 51 to the LEDs are preferably made at the top of the lighting systems 3 so that the connections are above the water.

In some embodiments of the invention, one or more further arrangements may be provided to prevent continuous exposure of algae to light emitted by the LEDs 20. One arrangement to reach such effect may be to provide a suitable movement of the aqueous liquid within the bioreactor tank. Additionally or alternatively, a swirling motion may be introduced in the tank, such that at different instants different portions of the algae are exposed.

Instead or in addition to suitable movement of the aqueous liquid comprising the algae, the LEDs 20 may be cycled on and off to accomplish discontinuous exposure. As a result of the discontinuous exposure caused by the suitable movement of the aqueous liquid and/or the on/off-cycle of the LEDs 20, carbon fixation in the algae may increase.

In order to force movement of the aqueous liquid within the bioreactor tank 1, a flow may be induced by means of injecting liquid at suitable positions, hereafter referred to as injection points. The injection points may be located in the bottom of the tank (bottom flow enhancers) and in the wall of the tank (side flow enhancers). For the flow enhancers placed under an angle in the wall, the angle is such that an upward flow is achieved.

Preferably, the liquid flow is added at an elevated pressure of 1-15 bars (per surface). In this way, the pressure difference between the main flow and the locally introduced extra liquid flow may affect the motion of the algae. The additional liquid flow may be adjustable to the viscosity of the aqueous liquid with algae If required, a pumping system can be used to deliver the additional liquid flow with a specific flow rate and with a specific density and viscosity.

In an embodiment, the pumping system is a disc pump. A disc pump is a pump comprising one or more discs to perform the pumping action. Due to the use of discs, damage to algae is avoided.

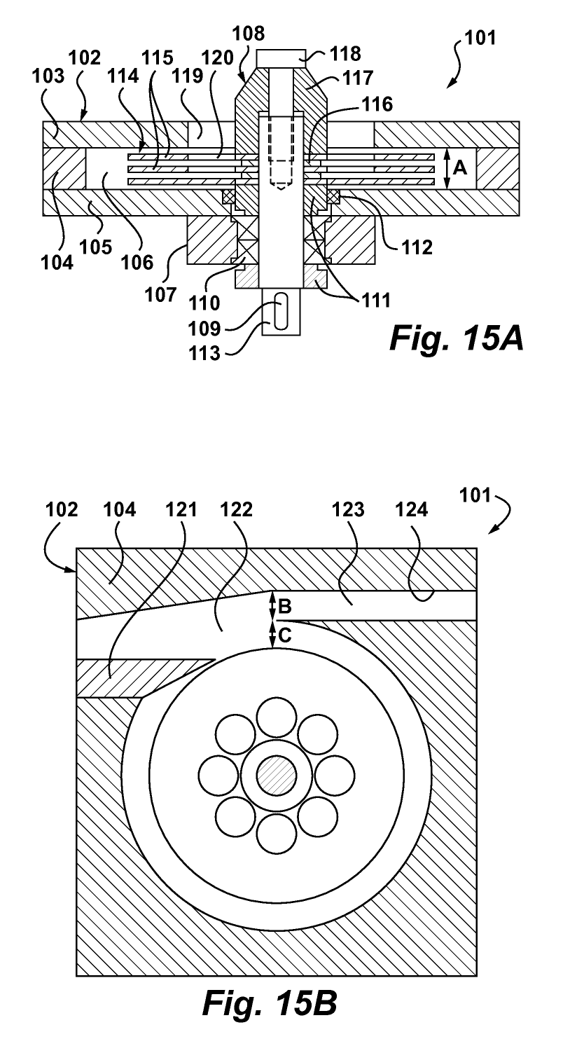

FIG. 15A shows a cross-sectional view of an embodiment of a disc pump. FIG. 15B shows a longitudinal sectional view of the same pump. The pump 101 comprises a housing 102 comprising a front plate 103, an intermediate plate 104 and a rear plate 105. The plates made be made of steel or of a plastic. The plates may be pressed together by bolts or the like (not shown). The intermediate plate 104 is provided with a circularly cylindrical recess, which, together with the front plate 103 and the rear plate 105, defines a chamber 106. The rear plate 105 comprises a bearing housing 107, in which a composite shaft 108 is rotatably accommodated by means of two bearings 110, e.g. double-seal ball bearings. The bearings 110 are clamped between two internally threaded rings 111, the inner ring 111 of which is sealed by a ring-shaped gasket 112. The shaft 108 is provided with a keyway 109, by means of which the shaft 108 can be connected to a drive unit, such as an electric motor.

Mounted on the central portion 113 of the shaft 108 is a rotor 114 which comprises a number of flat, round discs 115. The discs may be made of steel, stainless steel or a plastic, such as PVC or polycarbonate. The discs 115 are separated from each other by means of ring-shaped spacers 116. Additionally, the discs are pressed against the inner ring 111 by means of a clamping piece 117. In its turn, the clamping piece is mounted over the central portion 113 of the shaft 108 by means of a bolt 118. The discs 115 and the chamber 106 together form a so-called Tesla pump. Details of the design and operation of Tesla pumps are provided in U.S. Pat. No. 1,061,142 which is hereby incorporated by reference in its entirety. The larger the surface area and/or the number of discs, the larger the delivery and the propelling force of said pump will be.

The front plate 103 comprises a circular opening which fits over the clamping piece 117, forming an annular, axial inlet 119 therewith. As FIG. 16A shows, the discs 115 may be provided with a number of holes 120. Furthermore, a wedge-shaped insert 121 is mounted in the housing 102, which insert forms an outlet channel 122 together with the front plate 103, the intermediate plate 104 and the rear plate 105.

The pump is provided with a substantially tangential bypass channel 123, a first end of which opens into the outlet channel 122 of the pump 101, and a second end of which forms an inlet 124. The bypass channel 123 is formed in the intermediate plate 104 and has the same width A as the chamber 106. In order to ensure that the flow from the chamber is powerful enough to generate a significant flow through the bypass channel 123, the height B of the channel 123 at the outlet channel 122 is equal to or smaller than the distance C between an imaginary line transversely to the periphery of the rotor 114 and the internal wall of the chamber 106, likewise at the outlet channel 122.

The bypass channel 123, may be provided with an inlet for supplying carbon dioxide gas to the aqueous liquid. By supplying carbon dioxide gas in this matter, the size of carbon dioxide bubbles is very small. Such small CO₂-bubbles cause minimal damage to the algae.

The invention has been described by reference to certain embodiments discussed above. It should be noted various constructions and alternatives have been described, which may be used with any of the embodiments described herein, as would be know by those of skill in the art. Furthermore, it will be recognized that these embodiments are susceptible to various modifications and alternative forms well known to those of skill in the art without departing from the spirit and scope of the invention. Accordingly, although specific embodiments have been described, these are examples only and are not limiting upon the scope of the invention, which is defined in the accompanying claims.

1. A method for transferring light generated by a light emitting diode towards an aqueous liquid containing algae, the method comprising:
emitting light by the light emitting diode, the light emitting diode having a first refractive index;
transferring the light through a medium having a second refractive index;
further transferring the light through a solid medium having a third refractive index; and
passing the light into the aqueous liquid, the aqueous liquid having a fourth refractive index;
wherein the value of the first refractive index is equal to or greater than the value of the second refractive index, the value of the second refractive index is equal to or greater than the value of the third refractive index, and the value of the third refractive index is equal to or greater than the value of the fourth refractive index.

2. The method of claim 1, wherein the first refractive index has a value between 2.2 and 3.3.

3. The method of claim 2, wherein the medium is a cooling fluid, and the second refractive index has a value between 1.5 and 1.7.

4. The method of claim 1, wherein the medium is a cooling fluid, and the second refractive index has a value between 1.5 and 1.7.

5. The method of claim 1, wherein the solid medium comprises glass with a refractive index of 1.3 or higher.

6. The method of claim 1, wherein the light generated by the LEDs solely passes through the medium and the solid medium before passing into the aqueous liquid.

7. The method of claim 1, further comprising reflecting within the medium at least a portion of the light generated by the LEDs towards the solid medium.

8. The method of claim 7, wherein said reflecting is such that reflecting light is emitted substantially at right angles to the surface of the solid medium.

9. A reactor for growing algae in an aqueous liquid using photosynthesis, the reactor comprising:
a tank for accommodating the aqueous liquid with the algae in it; and
a lighting system being, at least partially, submerged in the aqueous liquid;
wherein the lighting system comprises a light emitting diode, a mounting structure for supporting the light emitting diode, and a housing for accommodating the light emitting diode and the mounting structure;
wherein the light emitting diode has a first refractive index;
wherein the housing is, at least partially, filled with a medium having a second refractive index;
wherein at least a portion of the housing is made of a solid medium that is transparent for light emitted by the light emitting diode, the solid medium having a third refractive index; and
wherein the aqueous liquid has a fourth refractive index;
wherein the value of the first refractive index is equal to or greater than the value of the second refractive index, the value of the second refractive index is equal to or greater than the value of the third refractive index, and the value of the third refractive index is equal to or greater than the value of the fourth refractive index; and
wherein the reactor is designed to transfer light emitted by the light emitted diode towards the aqueous liquid containing algae through the medium and the solid medium.
